(19) United States
(12) Patent Application Publication
(10) Pub. No.: US 2025/0186088 A1
(43) Pub. Date: Jun. 12, 2025

(54) GUIDED GROWTH SPINAL IMPLANTS

(71) Applicant: OrthoPediatrics Corp., Warsaw, IN (US)

(72) Inventors: Richard Detlefsen, Warsaw, IN (US); Matthew Prygoski, North Liberty, IN (US); Brian Snyder, Westwood, MA (US); James Sanders, Chapel Hill, NC (US); David Bumpass, Little Rock, AR (US); Richard M. Schwend, Kansas City, MO (US); Scott Luhmann, Ladue, MO (US); Richard McCarthy, Little Rock, AR (US)

(73) Assignee: OrthoPediatrics Corp., Warsaw, IN (US)

(21) Appl. No.: 18/686,190

(22) PCT Filed: Aug. 26, 2022
(86) PCT No.: PCT/US2022/075524

§ 371 Date: Feb. 23, 2024

Related U.S. Application Data
(60) Provisional application No. 63/237,371, filed on Aug. 26, 2021.

(51) Int. Cl. A61B 17/70; A61B 17/00

(52) U.S. Cl. CPC A61B 17/7035; A61B 2017/00526

(57) ABSTRACT

An orthopedic apparatus comprises an implantable anchor configured to retain an implantable rod and to permit translational movement of the rod relative to the anchor. A method for manufacturing an implantable rod comprises configuring the rod to facilitate translational movement of the rod relative to an implantable orthopedic anchor. An implantable anchor assembly includes a head that is adapted and configured to be supported by a connector assembly that is attached to a bone, such as a vertebrae: A rod extends through an enclosed rod pathway in a manner that permits the rod to slide relative to pathway, but at the same time place constraints on the rod as to lateral or vertical movement by head and rod retainer. Portions of the rod pathway are provided with a hard coating, such as ADLC.

### CROSS REFERENCE TO RELATED APPLICATION

This application claims the benefit of priority to U.S. Provisional Patent Application Ser. No. 63/237,371, filed Aug. 26, 2021, which is incorporated herein by reference.

### FIELD OF THE INVENTION

The inventions shown herein pertain to apparatus and methods for coupling a rod to a spine, and more specifically for couplings that permit sliding of the rod relative to an implanted anchor, and still further to those embodiments in which the anchor is adapted and configured to permit internal sliding passage of curved rods . . .

### BRIEF DESCRIPTION OF THE DRAWINGS

Some of the figures shown herein may include dimensions. Further, the figures shown herein have been created from scaled drawings, scaled models, or from photographs that are scalable. It is understood that such dimensions, or the relative scaling within a figure, are by way of example, and not to be construed as limiting unless so stated in a claim. Persons of ordinary skill will also recognize that CAD renderings may include lines that pertain to changes in the computer model, and not necessarily to component features.

FIG. 1 is a side, top perspective view of an assembled implantable anchor assembly according to one embodiment of the present invention.

FIG. 2 is a side elevational, partly exploded view of the apparatus of FIG. 1.

FIG. 3 is a side, perspective, partly exploded view of the apparatus of FIG. 1.

FIG. 4 is a front cross-sectional exploded view of the apparatus of FIG. 1.

FIG. 5 is a side, elevational exploded view of the apparatus of FIG. 1.

FIG. 6 is a top plan view of a portion of the apparatus of FIG. 1.

FIG. 7 is a side elevational view of the apparatus of FIG. 6, presented orthogonally to FIG. 6.

FIG. 8 is a side, top, perspective, cross-sectional view of the apparatus of FIG. 6.

FIG. 9 is a front elevational cross-sectional view of the apparatus of FIG. 1.

FIG. 10 is a side elevational view of a modified version of the anchor of FIG. 1.

FIG. 11 is a side elevational view of a modified version of the anchor of FIG. 1.

FIG. 12 is a side elevational view of a modified version of the anchor of FIG. 1.

FIG. 13 is a top plan view of an assembled set screw and rod retainer according to another embodiment of the present invention.

FIG. 14 is a side elevational view of the embodiment of FIG. 13, presented orthogonally to FIG. 13.

FIG. 15 is a front elevational view of the embodiment of FIG. 13.

FIG. 16 is a top plan view of the embodiment of FIG. 13, with the screw and retainer separated.

FIG. 17 is a side, top perspective view of an assembled implantable anchor assembly according to another embodiment of the present invention.

FIG. 18 is a top plan view of the embodiment of FIG. 17.

FIG. 19 is a front elevational cross-sectional view of a portion of the embodiment of FIG. 17, presented orthogonally to FIG. 18.

FIG. 20 is a side elevational cross-sectional view of a portion of the embodiment of FIG. 17, presented orthogonally to FIG. 18.

FIG. 21A is a side, top perspective, cross-sectional view of a head according to an embodiment of the present invention.

FIG. 21B is a front elevational view of the head of FIG. 21A.

FIG. 21C is side, cross-sectional view of the head of FIG. 21B, taken along line 21C-c of FIG. 21B.

FIG. 21D is a side, top perspective, cross-sectional view of a portion of an implantable anchor assembly according to another embodiment of the present invention.

FIG. 21E is a side, top perspective view of a portion of the embodiment of FIG. 21D.

FIG. 22A is a side, top perspective, cross-sectional view of a portion of an implantable anchor assembly according to another embodiment of the present invention.

FIG. 22B is a side, top perspective view of a portion of the embodiment of FIG. 22A.

FIG. 23A is a side, top perspective, cross-sectional view of a portion of an implantable anchor assembly according to another embodiment of the present invention.

FIG. 23B is a side elevational, partly exploded view of the apparatus of FIG. 23A.

FIG. 23C is a side, top perspective, assembled view of the apparatus of FIG. 23B.

FIG. 24 is a schematic of a rod according to an embodiment of the present invention.

FIG. 25 is a schematic of a rod according to another embodiment of the present invention.

FIG. 26 is a schematic of a rod according to yet another embodiment of the present invention.

### ELEMENT NUMBERING

The following is a list of element numbers used with all of the embodiments, and at least one noun used to describe that element. Consistent with statements made elsewhere in this specification, these various 2-digit element numbers are used among multiple embodiments, and aspects of a particular element stated for one embodiment can be applied to the same element number in a different embodiment, except as shown and described differently, and as would be understood by a person of ordinary skill in the art. It is understood that none of the embodiments disclosed herein are limited to these nouns, and these element numbers can further include other words that would be understood by a person of ordinary skill reading and reviewing this disclosure in its entirety.

| | |
|---|---|
| 10 | Bone |
| 20 | implantable anchor assy. |
| 24 | rod pathway |
| a | entrance / exist |
| b | central passage |
| c | minimum entrance / exit diameter |
| d | minimum central diameter |
| e | clearance |
| 28 | Bone connector assembly |
| a | captured end |
| b | Curved outer surface |
| c | Threaded shaft |
| d | cannula |
| e | Stop surface |
| f | central axis |
| 29 | bone connector coupling |
| a | ring |
| b | groove |
| c | flexible coupling wire |
| 30 | head |
| 31 | arm |
| a | rotational travel stop / abutment surface |
| b | vertical travel stop |
| 34 | Lower rod pathway |
| a | Entrance / exit |
| b | lower central passage |
| 35 | threads |
| 37 | pocket for connector assembly |
| 38 | aperture |
| a | bottom |
| b | top |
| 39 | Tool grasping features |
| 50 | Rod retainer |
| 51 | arm |
| a | rotational travel stop / abutment surface |
| b | vertical travel stop |
| 54 | Upper Rod pathway |
| a | entrance / exit |
| b | upper central passage |
| c | retainer height |
| d | retainer / head gap |
| 56 | channel for screw |
| a | bottom surface |
| b | overextending lip |
| 57 | screw capture features |
| a | weakened section; living hinge |
| b | material removal; wire cut |
| c | flexible wing |
| 58 | projection |
| 59 | Lower rod contact hoop |
| 80 | Set screw |
| 85 | threads |
| 88 | aperture |
| 90 | rod |
| 91 | end |
| 92 | outer surface |
| 93 | intermediate portion of rod |
| 94 | rough portion |
| 95 | smooth portion |
| 97 | hard coated portion |

### DETAILED DESCRIPTION OF ONE OR MORE EMBODIMENTS

At least one specification heading is required. Please delete this heading section if it is not applicable to your application. For more information regarding the headings of the specification, please see MPEP 608.01(a).

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”.

The use of an N-series prefix for an element number (NYY.YY) refers to an element that is the same as the non-prefixed element (YY.YY), except as shown and described. As an example, an element 1020.1 would be the same as element 20.1, except for those different features of element 1020.1 shown and described. Further, common elements and common features of related elements may be drawn in the same manner in different figures, and/or use the same symbology in different figures. As such, it is not necessary to describe the features of 1020.1 and 20.1 that are the same, since these common features are apparent to a person of ordinary skill in the related field of technology. Further, it is understood that some features 1020.1 and 20.1 may be backward compatible, such that a feature of a later discussed embodiment (NYY.YY) may include features compatible with other various embodiments that were discussed earlier (MYY.YY), as would be understood by those of ordinary skill in the art. This description convention also applies to the use of prime (′), double prime (″), triple prime (′″) and star or asterisk (*) suffixed element numbers. Therefore, it is not necessary to describe the features of 20.1, 20.1′, 20.1″, 20.1′″ and 20* that are the same, since these common features are apparent to persons of ordinary skill in the related field of technology.

Various references may be made to one or more methods of manufacturing. It is understood that these are by way of example only, and various embodiments of the invention can be fabricated in a wide variety of ways, such as by casting, sintering, sputtering, welding, electrodischarge machining, milling, as examples. Further, various other embodiment may be fabricated by any of the various additive manufacturing methods, some of which are referred to 3-D printing.

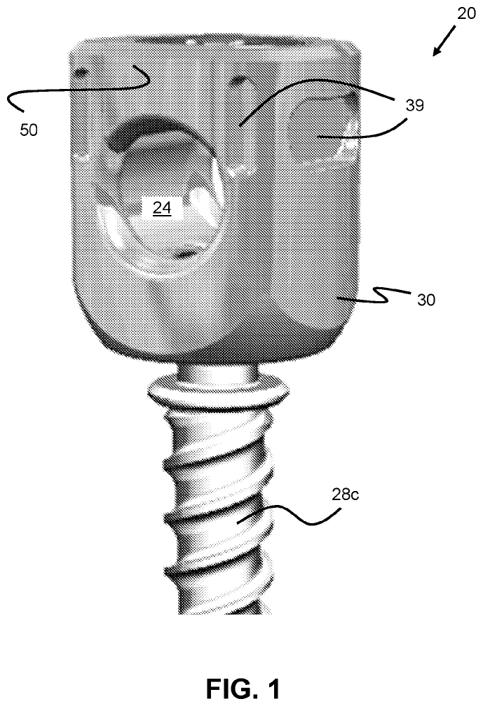

FIGS. 1-9 show various views of an implantable anchor assembly 20 according to one embodiment of the present invention. Anchor 20 includes a head 30 that is adapted and configured to be supported by a connector assembly 28 that is attached to a bone, such as a vertebrae. Preferably, a rod (not shown) extends through an enclosed rod pathway 24 in a manner that permits the rod to slide relative to pathway 24, but at the same time place constraints on the rod as to lateral or vertical movement by head 30 and rod retainer 50. In some embodiments, all or portions of the rod pathway are provided with a hard coating, such as ADLC. It is understood that the directions vertical, horizontal, lateral, and the like are with respect to the figures.

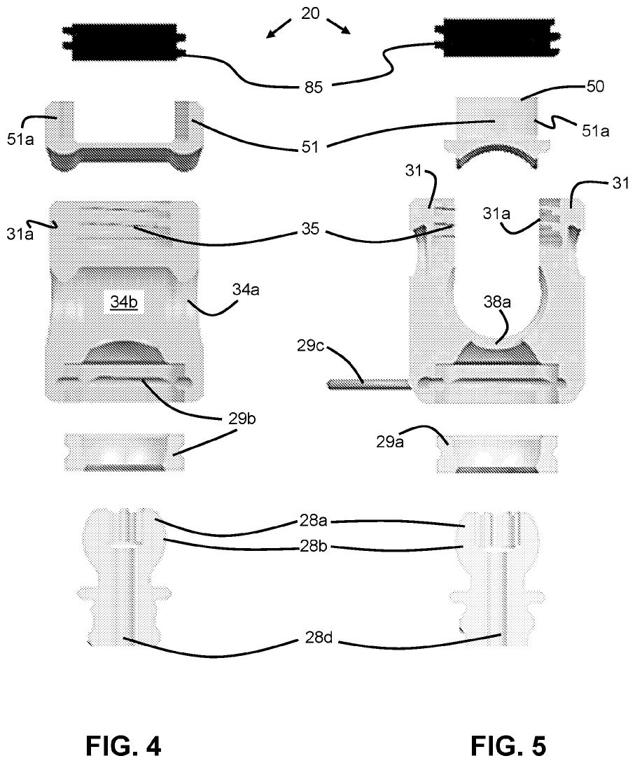

The connection of head 30 to bone connector 28 is best seen in FIGS. 4, 5, and 9. In one embodiment, connector assembly 28 includes a captured end 28a having a curved outer surface 28b. In some embodiments, surface 28b cooperates with connector coupling 29 and aperture 38 of head 30 to permit polyaxial rotation of head 30 relative to connector 28. In some embodiments, one or both of the captured end of the connector or surfaces in contact with the captured are provided with a hard coating, such as ADLC.

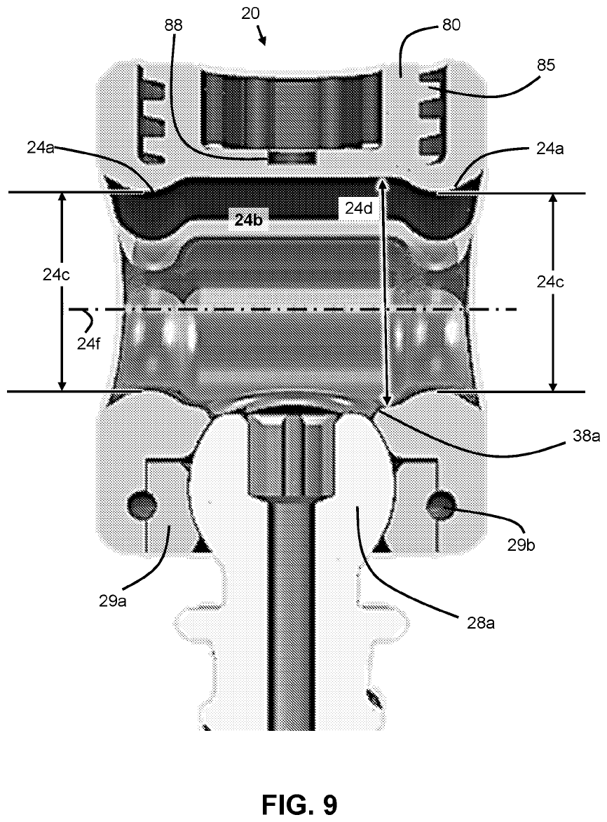

In some embodiments, connector coupling 29 includes a split ring 28a that fits generally around the bottom of the captured end 28a. The connector coupling ring 29a includes a partial groove 29b that cooperates with a corresponding groove in head 30 to form an open channel that can receive a flexible coupling wire 29c (inserted through an aperture in head 30), which locks ring 29a in the bottom of anchor assembly 20 as best seen in FIG. 9.

Although what has been shown and described is a rod-receiving head 30 coupled to a bone screw 28, yet other embodiments of the present invention contemplate yet other means for connection to a bone, including: curved outer surfaces with flat ends, such that uniaxial pivoting is permitted; rigid fixation of a bone screw or other device to the head; attachment of the head to a connector assembly that connects to a bone by way of a strap that extends around the bone; as non-limiting examples.

Some embodiments of the present invention further include a stop ring or stop surface 28e that prevents over tightening of the bone connector, such as overtightening to the point where the bottom surface of the head 30 comes into contact with the bone outer surface. The use of a stop ring thus permits a range of polyaxial movement of the head relative to the bone connection device, without any hindrance by contact with the bone surface.

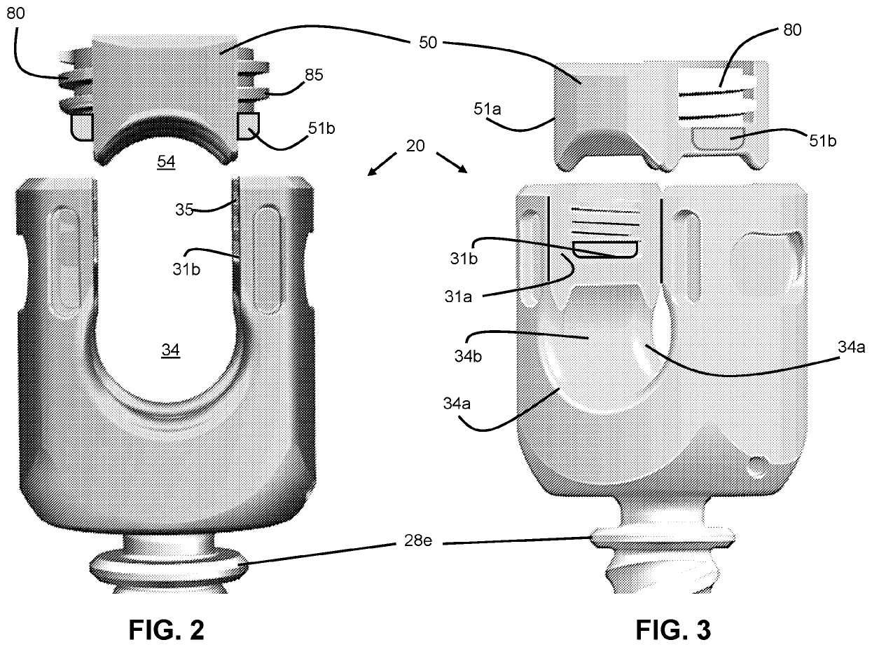

FIGS. 2 and 3 show a partially exploded anchor assembly 20, with a partial assembly of a set screw 80 within a rod retainer 90 spaced apart from threaded connection with head 30.

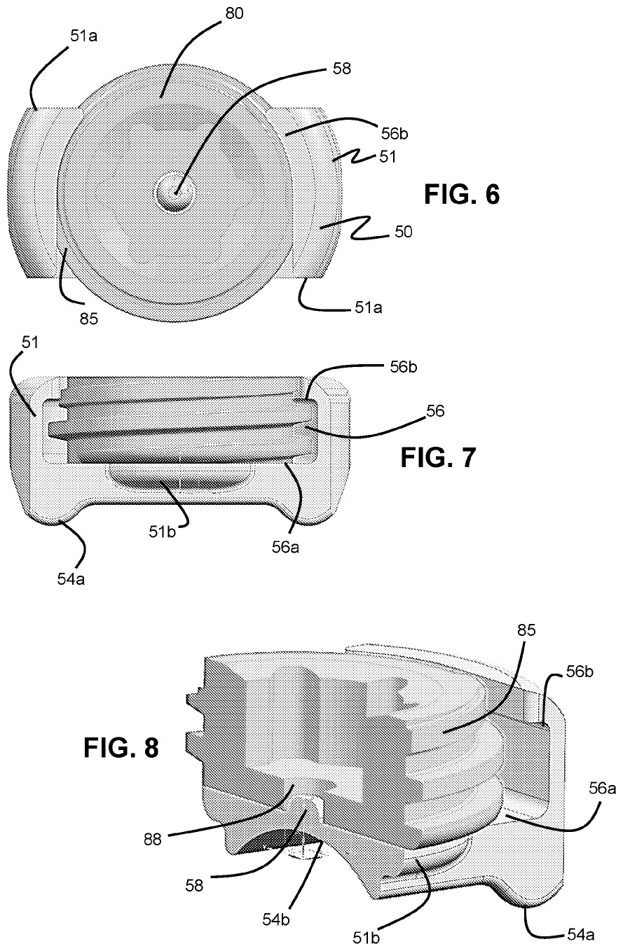

Referring briefly to FIGS. 6, 7, and 8, it can be seen that the upper assemblies shown in FIGS. 2 and 3 include a set screw 80 that is received within a channel 56 of rod retainer 50. Channel 56 is preferably formed between a bottom surface 56a of retainer 50 and an overextending lip 56b. The bottom of set screw 80 rests upon bottom surface 56a. The threads 85 of set screw 80 have a major diameter that fits within channel 56, but which is too large to pass by the lip 56b (best seen in FIG. 7).

Referring to FIGS. 6 and 8, it can be seen that the set screw 80 includes a bottom aperture 88 that receives within it a projection from bottom surface 56a. Projection 58 assists in capturing screw 80 on retainer 50, and also providing a general centering of screw 80 on retainer 50. Screw 80 is rotatable relative to retainer 50 about projection 58.

It is to be noted that screw 80 is free to rotate within slot 56, referring to FIGS. 6 and 8. Therefore, as threads 85 engage threads 35, the rotation of the set screw in a tightening direction results in the bottom of the set screw pushing against bottom surface 56a of channel 56. Likewise, loosening of screw 80 results in rotation of threads 85 against the bottom of overextending lip 56b of retainer 50, thereby lifting the assembly of the screw and retainer.

Referring to FIG. 6, retainer 50 includes a pair of arms 51 that extend upwardly from the bottom of retainer 50. In some embodiments, one or both of arms 51 are adapted and configured to cooperate with head 30 so as to prevent rotation of retainer 50 relative to head 30. Referring to FIGS. 5 and 6, it can be seen that retainer 50 includes at least one surface 51a that is adapted and configured to come into abutting contact with a corresponding rotational travel stop 31a of head 30. In the embodiment shown, retainer 50 includes four (4) rotational travel stops 51a, each of which is adapted and configured to achieve abutment of at least one pair of surfaces 51a with a corresponding surface 31a of head 30. However, yet other embodiments of the present invention contemplate the interaction of only two features (one on the retainer, the other on the head) to restrain rotation of retainer 50 relative to head 30. Further, configurations other than the abutment of opposing surfaces are contemplated, such as a projection or pin that is received within a corresponding slot, or other configurations.

FIGS. 2, 3, and 7 show various aspects of means for preventing compression of the rod according to one embodiment of the present invention. Rod retainer 50 includes one or more vertical travel stops 51b that project outwardly from one or more side or lateral surfaces of the retainer 50. These vertical travel stops 51b are adapted and configured to coact with vertical travel stops 31b of arms 31 of head 30. Referring to FIG. 3, it can be seen that as the partial assembly of retainer 50 and set screw 80 is placed between arms 31, that the laterally projecting travel stops 51b (as best seen in FIG. 2) are aligned generally over top of the vertical travel stops 31b.

As the set screw and retainer pass between arms 31, the threads 85 engage the threads 35. Referring to FIG. 2, it can be seen that the maximum lateral extent of threads 85 (i.e., the major diameter) extends laterally outward further than the lateral most extent of a vertical travel stop 51b. Further, the major diameter of the internal threads 35 of arm 31 have a lateral extent (or diameter) that is greater than the lateral most extent of a vertical stop 51b. Therefore, travel stops 51b do not interfere with engagement of threads 85 with threads 35. After engagement of threads 85 and 35, continued tightening of set screw 80 will result in a lowering of retainer 50 into head 30 (since set screw 80 is captured within channel 56). The maximum vertically downward displacement of rod retainer 50 between arms 31 is limited by abutting contact between vertical stops 51b and 31b.

Still further embodiments of the present invention contemplate the usage of set screws and retainers that are adapted and configured to permit an adjustable clearance from the rod to the retainer. As one example, the threads 35 and 85 can be provided with fine pitch threads that resist backout because of the shallowness of the threads. the surgeon can adjust the set screw to the desired rod-retainer clearance, and the shallow threads resist backing our from that position. Alternatively, a second set screw could be added to the head after the first set screw is tightened to the desired rod clearance, with the second set screw being locked against the first set screw to prevent backout.

As another alternative. the threads of such a set screw could be established with a small degree of interference with the threads of the head, such that the threads resist back out by the friction developed by the interference. In yet another embodiment, the retainer and/or head are provided with tapered threads, such as tapered threads in accordance with NPT or BSP standards. In such threads (or any of the threads described herein), it may be desirable to increase the hardness of the threads (such as by application of a hard coating) to minimize any generation of particles by the threaded coupling.

In still further embodiments, the adjustability of the rod clearance can be achieved by placing a deformable device (deformable elastically or inelastically) between the travel stops 51b and 31b. Examples of such devices include a crushable seal, a hollow crush washer, Belleville washer, wave spring, or lock washer). As the set screw is tightened, the cross sectional height of the washer is reduced. Application of increased torque results in increased height reduction and decreased rod clearance.

Although what has been shown and described are apparatus and methods for limiting the downward movement of retainer 50 by way of a pair of opposing travel stops 51b that contact a pair of opposing travel stops 31b to limit retainer travel, yet other apparatus and methods are contemplated by this invention. It is to be noted that several different surfaces of retainer 80 can be adapted and configured to coact with other features of head 30 to provide a limit to the movement of retainer 50. For example, one or more of arms 31 could include one of a projection or slot that coacts with the other of a projection or slot on retainer 50, such that alignment of the projection and the slot results in travel-limiting contact of retainer 50 relative to head 30, as one non-limiting example.

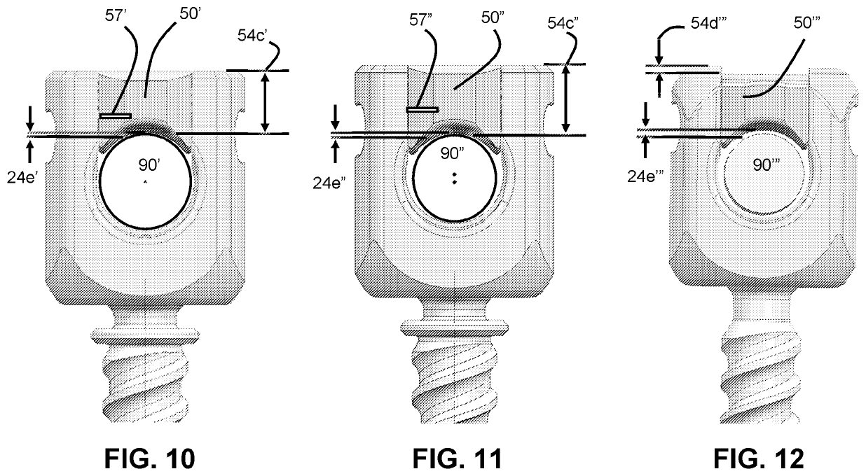

The assembly of retainer 50 onto head 30 results in a closure of lower rod pathway 34 by the upper rod pathway 54, which results in establishment of a the rod pathway 24. Referring to FIGS. 10-12, it can be seen that in some embodiments the clearance gap 24e of the rod pathway 24 can be established by selection of the rod diameter and the configuration of the vertical travel stops of rod retainer 50 and head 30. FIG. 10 includes a retainer 50′ engaged with and bottomed out within a head, and capturing a rod 90′. As one example, rod 90′ is six millimeters in diameter, and the height 54c′ of retainer 50 (from the top surface of the retainer to the closest point of the arc of the upper rod pathway) that is four millimeters. Note that retainer 50′ includes a screw capture feature 57′ that will be described later. The embodiment of FIG. 10 shows that tightening of set screw 80′ results in a rod-to-retainer clearance 24e′.

The assembly of retainer 50 onto head 30 results in a closure of lower rod pathway 34 by the upper rod pathway 54. Preferably, closure of the pathway does not necessarily mean that the retainer frictionally clamps to the rod. Referring to FIGS. 10-12, it can be seen that in some embodiments the clearance gap of 24e of the rod pathway can be established by selection of the rod diameter and the relative placements of the vertical travel stops. FIG. 11 includes a retainer 50″ engaged with and bottomed out within a head, and capturing a rod 90″. As one example, rod 90″ is five and one-half millimeters, and the height 54c″ of retainer 50 (from the top surface of the retainer to the closest point of the arc of the upper rod pathway) that is four and one-half millimeters. Note that retainer 50″ includes a screw capture feature 57″ that will be described later. The embodiment of FIG. 11 shows that tightening of set screw 80″ results in a rod-to-retainer clearance 24e″.

The assembly of retainer 50 onto head 30 results in a closure of lower rod pathway 34 by the upper rod pathway 54. Referring to FIGS. 10-12, it can be seen that in some embodiments the clearance gap of 24e of the rod pathway can be established by selection of the rod diameter and the configuration of the rod retainer 50. FIG. 12 includes a retainer 50′″ engaged with and bottomed out within a head, and capturing a rod 90′″. As one example, rod 90′″ is five and one-half millimeters, and the height 54c′″ of retainer 50 (from the top surface of the retainer to the closest point of the arc of the upper rod pathway) that is one-half millimeters. The embodiment of FIG. 12 shows that tightening of set screw 80′″ results in a rod-to-retainer clearance 24e′″. It can be seen that the vertical travel stops 51b′″ and 31b′″ of FIG. 12 are established such that the upper surface of the retainer includes a gap 54d′″.

One feature of some embodiments of the present invention is to create a rod pathway 24 that provides clearance (or looseness) between the rod and the assembled anchor. Referring to FIG. 9, it can be seen that an assembled implantable anchor 20 includes at least one of an entrance or exit for the rod 24a, with a larger interior central passage 24b located between the entrance and exit. As shown in FIG. 9, in one embodiment each entrance and exit 24a has a minimum diameter 24c.

In some embodiments of the present invention, an implanted anchor X20 and rod X90 are part of a larger implanted array of devices. For example, in some embodiments there are multiple implantable anchor assemblies, one or more of which provide sliding capture of the rod (such as an anchor X20), and one or more of which provide frictional capture of a portion of the rod. For such frictional capture, the sliding retainer X50 of an anchor X20 can be replaced with a clamping retainer. The clamping retainer is adapted and configured to have an upper pathway that comes into contact with the outer diameter of the rod. As examples, the clamping retainer may not have a vertical travel stop 51b (alternatively, the clamping head may not include a vertical travel stop 31b), such that continued tightening of the set screw is largely unimpeded until contact is made between the rod and the retainer. Some embodiments of the present invention pertain to a kit of devices that includes an implantable anchor X20 and multiple retainers (both sliding and capturing). Still further embodiments contemplate kits of retainers (both sliding and capturing) that are adapted and configured for different diameters of rods and fit into the same head thread pattern 35.

The entrance and exit each lead to the central passage 24b that has a diameter (or for those embodiments in which central passage 24b is not circular in cross section, a minimum opening dimension) 24d that is greater than at least one of the entrance height 24c or the exit height 24c. As shown in FIG. 9, in one embodiment, entrance and exit 24a are preferably circular in cross section, and coaxial about a pathway centerline 24f. Preferably, the central passage 24b is further circular in cross section and coaxial with centerline 24f. Referring to FIG. 9, it can be seen that the cross sectional shape of rod pathway 24 is narrower at the entrance and exit, and larger intermediate of the entrance and exit. In this manner, pathway 24 can slidingly capture a rod that is bent, such as by receiving a bent portion of the rod through the entrance, to within the central passage 24b, and out through the exit. Although what has been shown and described are entrance, central, and exit passages that are coaxial and circular, yet other embodiments contemplate central passages that are offset from the axis 24f connecting the entrance and exit, and still further contemplate entrance, exit and central passages that have non-circular cross sectional shapes, including oblong, oval, and polygonal (preferably with rounded corners) cross sectional shapes.

FIG. 9 further shows that the entrance and exit include minimum diameter cross sections that are rounded and curved in order to better handle contact stresses resulting from movement of the rod after implantation into the patient. FIG. 2 illustrates that the central rod pathway 24 is established by the tightening the assembly of set screw 80 and rod retainer 50 into head 30. Rod retainer 50 includes an upper rod pathway 54 having and an entrance and exit 54a as well as an upper central passage 54b (as can also be seen in FIGS. 7 and 8). Likewise, head 30 defines a lower rod pathway 34 having an entrance and exit 34a, with a lower central passage 34b located between the entrance and exit. Referring again to FIG. 9, it can be seen that the assembly of retainer 50 onto head 30 results in general alignment of the entrances and exits 24a and 34a, and further general alignment of central passages 34b and 54b.

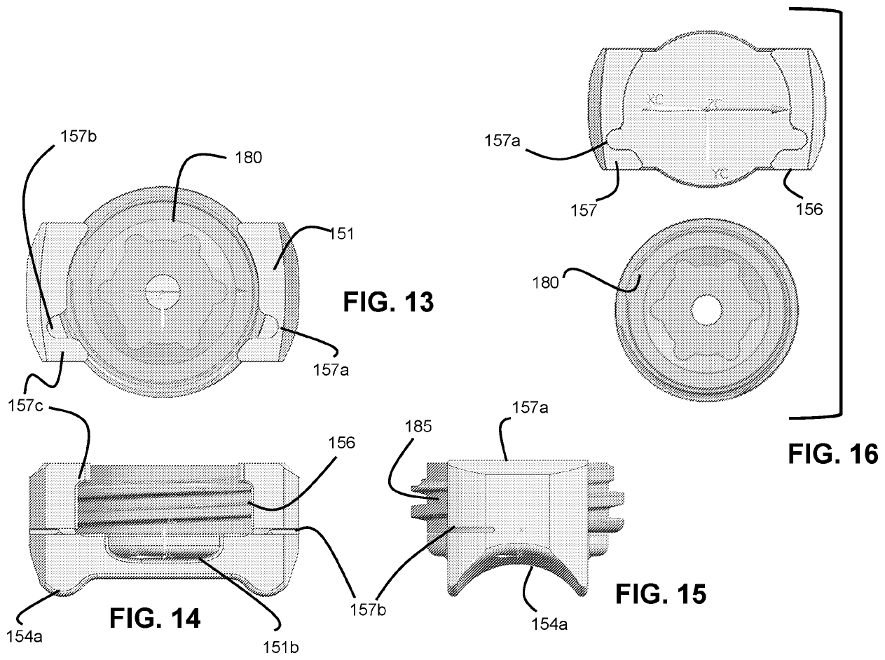

FIGS. 13-16 show various views of a retainer 150 according to another embodiment of the present invention. Rod retainer 150 includes means for capturing a set screw by the retainer.

Retainer 150 includes one or more capture features 157 that are adapted and configured to assist in placing the screw 180 into channel 156, and to make it difficult to remove a set screw 180 after it has been placed within channel 156. Referring to FIG. 13, it can be seen in this top view that the top surface of each arm 151 includes an open preferably semi-circular area 157b that assists in lowering the lateral stiffness of the top of channel 156 (lateral referring to bending in the plane of FIG. 13). In so doing, a weakened or living hinge 157a is formed in the top of the channel 156. The creation of this hinge 157a permits a small foot 157c to be elastically bent so as to create a temporarily larger entrance to the channel. As seen in FIG. 13, the flexible ears or wings 157c can be bent in the plane of the figure. Referring to FIG. 14, the ears 157c can elastically bend in a direction that is in and out of the plane of the figure.

FIGS. 14 and 15 further show another area of weakening and material removal 157b that extends within each opposing arm 151, and which further lowers the stiffness of the living hinge. In one embodiment, this removed area can be produced by way of an EDM wire cut. The removal of this material further reduces the amount of load required to bend the wings 157c out of the way when screw 180 is inserted.

FIG. 16 shows the screw and retainer separated. Screw 180 can be slid laterally into channel 156, with each of the wings 157c bending outwardly at the hinge 157a to permit entry of the screw into the channel. Once the screw is seated in the retainer, the elastic hinges return the wings back to their original position, thus capturing the screw within the rod retainer 150.

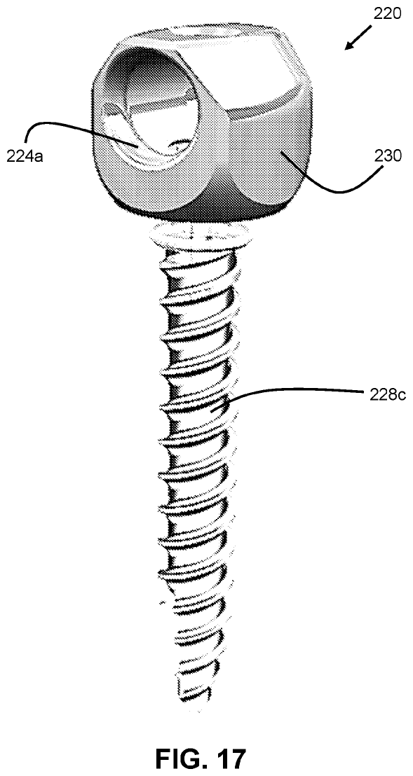

FIGS. 17-23 show various aspects of an implantable anchor assembly 220 according to another embodiment of the present invention. Anchor 220 is similar to anchor 20 as previously discussed, except that head 230 does not have a removable rod retainer and set screw, as will now be described.

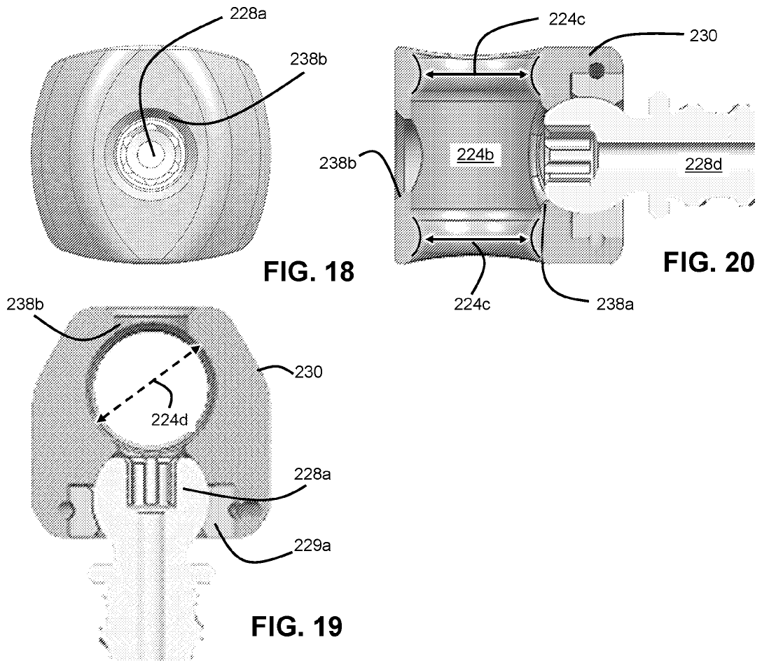

Referring to FIG. 18, it can be seen that the top surface of head 230 includes an aperture 238b that permits access from this top surface to the captured end 228a of connect assembly 228. As shown herein, the captured end 228a includes a pocket that is adapted and configured to mate with a complementary-shaped tool for tightening of connector 228 into the bone of the patient. Head 230 is preferably constructed in one piece.

As best seen in FIGS. 19 and 20, head 230 of assembly 220 includes entrances and exits 224a and a central passage 224b that are generally enclosed within a preferably one piece head 230. Referring to FIG. 20, it can be seen that each of the entrance and exit 224a have a minimum size of opening 224c (in some embodiments, a circular, inner diameter) that are smaller than the larger minimum passage dimension 224d of the central passage 224b.

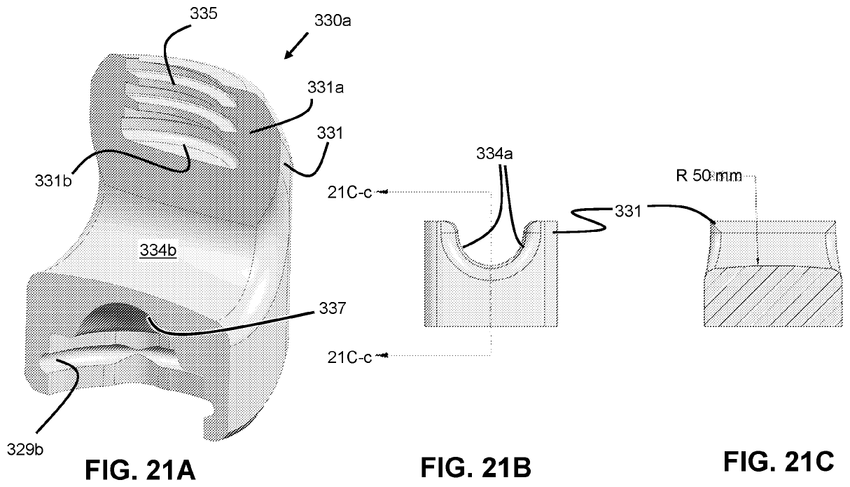

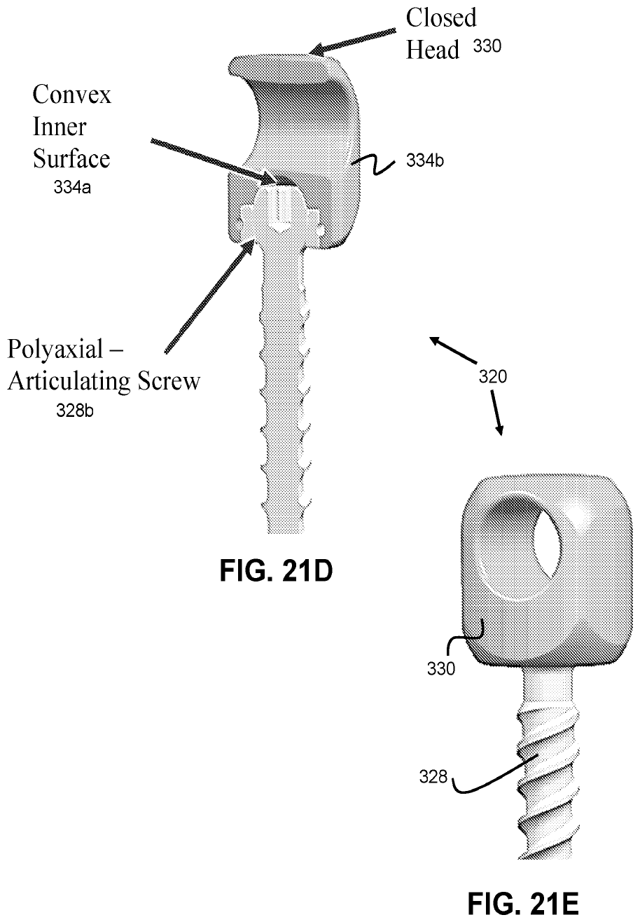

FIGS. 21A, 21B, and 21C show various aspects of a portion of a head 330a. In this embodiment, head 330a includes an enclosed pocket 337 in which the captured end 328a (such as a polyaxial head, not shown) of the bone connection assembly will reside. Head 330a does not include a bottom aperture 338b. It has been found in some embodiments that the placement of an aperture within the central passage (to permit access to the top of the connection device) is a source of stress concentration when the rod contacts the edges of the aperture. By removing this aperture, the contact stresses and subsequent wear will be minimized. In some embodiments the lower central passage has a radius parallel to the central axis of about forty to sixty millimeters, and preferably within the range of forty-five to fifty-five millimeters. However, yet other embodiments of the present invention contemplate use of a similar convex, but including a central passage for access to the screw head.

The use of a relatively shallow, hemispherical dome shape on the bottom of the set screws and at the base of the tulip provides a rod-to-head arrangement that may “articulate” with the rod as the rod changes its angle through the central pathway. In this manner, the shape of the bottom of the set screw and of the pathway acts to an extent as a ball bearing, and can accommodate a range of rod curvature. Note that the smallest passageway dimension of pathway 334b is in the middle of the passage, and that the entrance and exit 334a both include a radius that rolls off and downward, in contrast to the entrance and exit 24a (as best seen in FIG. 9) in which preferably each exit and entrance rolls upward to establish a minimum dimension 24c of the rod pathway.

FIGS. 21D and 21E show various aspects of an implantable anchor assembly 320 according to another embodiment of the present invention. Anchor 320 includes a head that is similar to the head shown in FIG. 21A, except that the head 330 is closed at the top. Head 330 is similar in some respects to head 230 as shown in FIG. 20, except that the minimum rod pathway dimension of head 230 would be the entrance/exit dimensions 224c, which are smaller than the interior pathway dimension 224d. In contrast, head 320 includes an interior pathway 324d that establishes the minimum dimension of rod pathway 324 of FIG. 21D. Both the entrance and exit include cross sectional shapes that roll outward from the centerline of the rod pathway, rather than the cross sectional shapes of the entrance of exit of head 230 which role inward toward the centerline.

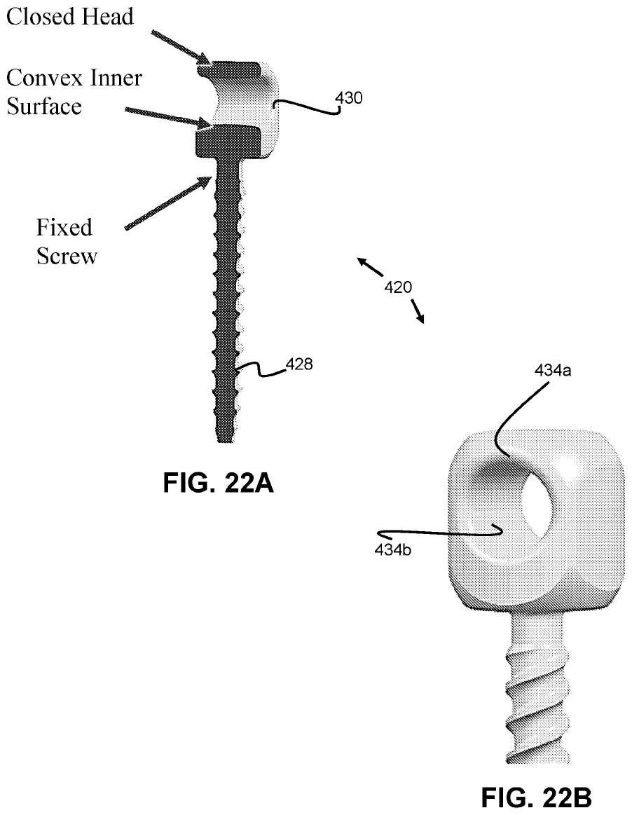

FIGS. 22A and 22B each include a head 436 similar to the head described for the device of FIGS. 21D and 21E, except that the screw 428 is fixed relative to head 436.

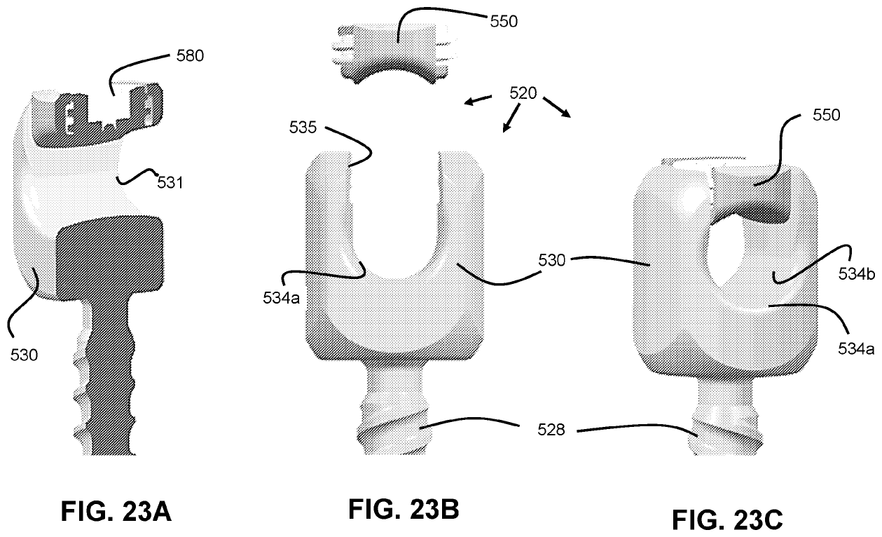

FIGS. 23A, 23B, and 23C show yet another anchor 520 that includes the threads 535 that accept the subassembly of set screw 580 and retainer 550, which is the same as the set screw and retainer shown in FIGS. 2 and 3. Anchor assembly 520 includes a head 530 that is fixed to bone connector 528, similar to the bone connector and head of FIGS. 22A and 22B.

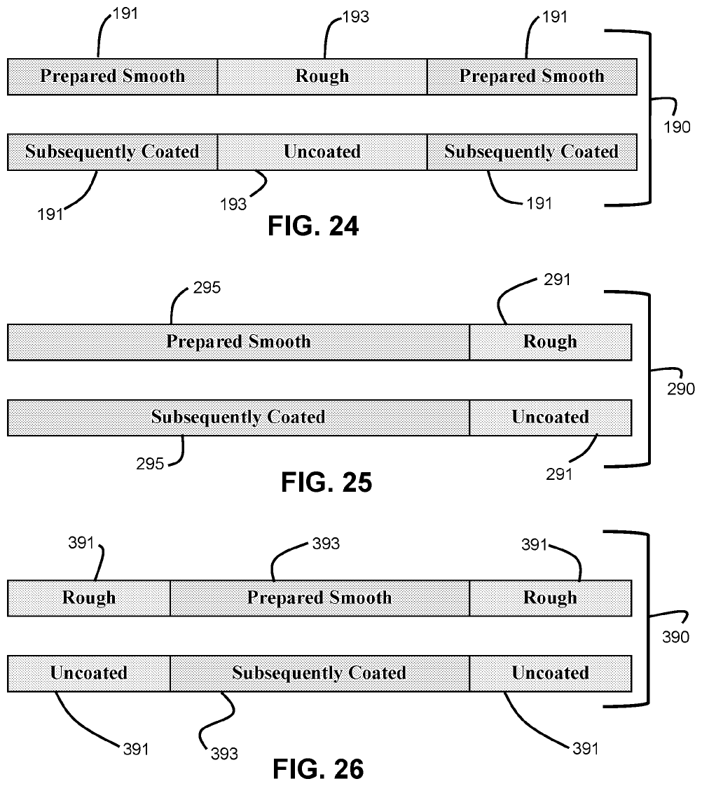

FIGS. 24-26 show various configurations of rods 190, 290, and 390, respectively, that are adapted and configured for use with one or more of the anchor assemblies X20 shown herein. As previously discussed, anchors X20 preferably do not frictionally clamp onto a rod passing within the pathway X24. Instead, these pathways X24 are adapted and configured for sliding contact, with the minimum dimension of the pathway being greater of the outer diameter of the rod. However, the rod in some embodiments is frictionally (or otherwise) connected to an implantable anchor, such that there is no planned relative movement between the rod and the clamping anchor assembly. This frictional clamping can occur at either end of the rod, or at a portion of the rod intermediate of the ends.

Each of the rods shown in FIGS. 24, 25, and 26 are shown schematically in two representations: one representation showing the arrangement of smooth or rough portions, and the other representation (of the same rod) showing portions treated with a hard, preferably low-friction coating such as an amorphous diamond-like coating (“ADLC”). However, the present invention is not limited to the use of ADLC, and contemplates other type of coatings and surface treatment to enhance the hardness and/lower the friction of the coated rod diameter, including as examples the use of an anodizing treatment or an application of a chromium coating.

In some embodiments, the surface of the rod preferably has at least one roughened portion and one smooth portion. Preferably, the smooth portion would have a surface roughness Ra of anywhere from about one one-hundredth of a micron to about one micron, as an example. The roughened portion of the rod has a surface finish preferably greater than one micron, and more preferably than about 20 microns. In some embodiments, the roughened surface is achieved by use of a media blast, such as by grit blasting, shot peening, glass bead blasting, or the like.

FIG. 24 shows a schematic of a rod 190 that includes a pair of opposing ends 191, each of which has been prepared smooth, and which are subsequently coated with ADLC. These ends would therefore slidingly support a vertebrae by use of one of the implants X20 shown herein. An intermediate portion 193 of the rod 190 is preferably not coated, and further preferably roughened. This intermediate portion 193 of the rod 190 is adapted and configured for frictional coupling to an implantable anchor assembly (not shown).

FIG. 25 shows yet another configuration of a rod 290, in which one end 291 of the rod 290 is intentionally roughened and left uncoated for frictional connection to an anchor. The remainder 295 of the rod 290 is prepared to be smooth, and coated.

FIG. 26 shows a rod 390 that has a topology that is generally opposite of the topology of rod 190. Each of the ends 391 are left uncoated, and intentionally roughened. This permits placement of rigid connections on the rod at each end. The intermediate portion 393 is prepared smooth, and subsequently coated. One or more anchors X20 would connect to this portion of the rod.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. An implantable apparatus for connecting a rod to a bone, comprising:
a set screw having a threaded outer surface;
a bone connection device having a rounded end;
a head having a first pair of opposing threaded arms and a portion of a rod pathway defined between the arms, said head further including a pocket for receiving the rounded end, said head being pivotal relative to said bone connection device in at least one direction, said head including a first travel stop; and
a rod retainer having a surface adapted and configured to enclose a portion of the rod pathway, said rod retainer having a second pair of opposing arms that define a channel therebetween, said rod retainer including a second travel stop;
wherein said rod retainer is slidably received between the first pair of opposing arms, said set screw is slidably received in the channel of the second pair of arms, and threaded engagement and tightening of said set screw in said first threaded arms moves said rod retainer relative to the rod pathway until the first travel stop and the second travel stop come into abutting contact.

2. An implantable apparatus for connecting a rod to a bone, comprising:
a bone connection device having a rounded end;
a head having an enclosed rod pathway having an entrance, and exit, and a central passage therebetween, said head further including a pocket for receiving the rounded end, said head being pivotal relative to said bone connection device in at least one direction; and
wherein the exit has a first cross-sectional area perpendicular to the pathway, the entrance has a second cross sectional area perpendicular to the pathway, and the central passage has a portion with a third cross sectional area perpendicular to the pathway, the third cross sectional area being greater than the first cross-sectional area and greater than the second cross sectional area.

3. A kit for coupling together multiple vertebra, comprising:
an implantable rod having a length and two ends and an outer surface between the two ends, a first portion of the surface along the length of said rod having a first surface roughness, a second portion of the surface along the length of said rod having a second surface roughness that is rougher than the first surface roughness;
an implantable first bone anchor adapted and configured for attachment to a vertebra, said first bone anchor including a first bone connection device, a first head having a first rod passageway adapted and configured to receive said rod and to permit sliding movement of said rod relative to said first head, said first head including means for preventing clamping of the first portion of said rod by said first head; and
an implantable second bone anchor adapted and configured for attachment to a vertebra, said second bone anchor including a second bone connection device and having a second head attached to said second bone connection device, said second head having a second rod passageway adapted and configured to receive said rod and to frictionally clamp the second portion of said rod to said second head.

4. A kit for coupling together multiple vertebra, comprising:
an implantable rod having length and two ends and an outer surface between the two ends, a first portion of the surface along the length of said rod having a first surface hardness, a second portion of the surface along the length of said rod having a second surface hardness that is harder than the first surface hardness;
an implantable first bone anchor adapted and configured for attachment to a vertebra, said first bone anchor including a first bone connection device, a first head having a first rod passageway adapted and configured to receive said rod and to permit sliding movement of said rod relative to said first head, said first head including means for preventing clamping of the first portion of said rod by said first head; and
an implantable second bone anchor adapted and configured for attachment to a vertebra, said second bone anchor including a second bone connection device and having a second head attached to said second bone connection device, said second head having a second rod passageway adapted and configured to receive said rod and to frictionally clamp the second portion of said rod to said second head.

5. A method for manufacturing an implantable rod, comprising:
providing a rod having two ends, a centerline, and a length therebetween:
preparing at least a first portion of the length along the centerline at one end to have a surface roughness less than about one micron Ra;
coating the surface of the prepared portion at the one end with a material adapted and configured to harden the surface; and
roughening the surface of a second portion of the length along the centerline intermediate of the two ends by media blasting.

6. The apparatus of claim 1, wherein tightening of said set screw results in bottoming contact of said rod retainer in the first pair of arms.

7. The apparatus of claim 1, which further comprises means for preventing rotation of said rod retainer relative to said head.

8. The apparatus of claim 2, wherein said head includes a first travel stop, and which further comprises a rod retainer having a second travel stop, and said first travel stop and said second travel stop contact each other to prevent clamping of a rod within the rod pathway by said rod retainer and said head.

9. The apparatus of claim 2, wherein the first cross sectional area has a first centroid, the second cross sectional area has a second centroid, the third cross sectional area has a third centroid, the first centroid and second centroid define a straight line going through the third cross sectional area, and the third centroid is offset from the straight line.

10. The apparatus of claim 2, wherein the first cross sectional area is circular, the second cross sectional area is circular, and the third cross sectional area is circular.

11. The apparatus of claim 2, wherein the first cross sectional area has a first center, the second cross sectional area has a second center, the third cross sectional area has a third center, and the first center, the second center, and the third center are colinear.

12. The kit of claim 3, further comprising a set screw threadably received by said second head, wherein tightening of said set screw can cause frictional clamping of the rod by the second head.

13. The kit of claim 3, further comprising a set screw threadably received by said second head, wherein tightening of said set screw results in application of a compressive load against the surface of said rod.

14. The kit of claim 3, wherein a hardening coating of amorphous diamond-like coating is on the rod.

15. The apparatus of claim 1, wherein at least one of an entrance of the rod pathway, an exit of the rod pathway, and a central passage of the rod pathway includes a hard coating.

16. The apparatus of claim 1, wherein at least one of the rounded end of the bone connection device and the pocket of the head receiving therein said bone connection device includes a hard coating.

17. (canceled)

18. (canceled)

19. The apparatus of claim 2 wherein said head includes a first pair of threaded arms, and which further comprises a set screw having a threaded outer surface which further comprises a rod retainer having a surface adapted and configured for contact with said set screw and to enclose a portion of the rod pathway, said rod retainer being slidably received within the first pair of arms, wherein tightening of said set screw results in bottoming contact of said rod retainer in the first pair of arms.

20. The kit of claim 3 wherein said first head includes a first pair of threaded arms, and which further comprises a set screw having a threaded outer surface, a rod retainer having a surface adapted and configured to enclose a portion of the rod pathway, said rod retainer being slidably received within the first pair of arms, wherein tightening of said set screw in the first pair of arms results in bottoming contact of said rod retainer in the first pair of arms.

21. The kit of claim 4 wherein said first head includes a first pair of threaded arms, and which further comprises a set screw having a threaded outer surface, a rod retainer having a surface adapted and configured to enclose a portion of the rod pathway, said rod retainer being slidably received within the first pair of arms, wherein tightening of said set screw in the first pair of arms results in bottoming contact of said rod retainer in the first pair of arms.

22. The apparatus of claim 2 wherein said head includes a threaded portion and a first travel stop, and which further comprises a threaded set screw and a rod retainer having a surface adapted and configured to enclose a portion of the third cross-sectional area and a second travel stop, said rod retainer being slidably received within the threaded portion of said head, said set screw being supported by said rod retainer, and abutting contact of said first travel stop and said second travel stop prevents rotation of said rod retainer relative to said head when said set screw is tightened in the threaded portion of said head.

23. The kit of claim 3 wherein said first head includes a threaded portion and a first travel stop, and which further comprises a threaded set screw and a rod retainer having a surface adapted and configured to enclose a portion of the first rod passageway and a second travel stop, said rod retainer being slidably received within the threaded portion of said first head, said set screw being supported by said rod retainer, and abutting contact of said first travel stop and said second travel stop prevents rotation of said rod retainer relative to said first head when said set screw is tightened in the threaded portion of said first head.

24. The kit of claim 4 wherein said first head includes a threaded portion and a first travel stop, and which further comprises a threaded set screw and a rod retainer having a surface adapted and configured to enclose a portion of the first rod passageway and a second travel stop, said rod retainer being slidably received within the threaded portion of said first head, said set screw being supported by said rod retainer, and abutting contact of said first travel stop and said second travel stop prevents rotation of said rod retainer relative to said first head when said set screw is tightened in the threaded portion of said first head.

25. The kit of claim 3, which further comprises a threaded set screw, wherein said means for preventing clamping includes a travel stop located in said head, said head includes a threaded portion to receive said set screw, and tightening of said set screw to the travel stop results in clearance of said rod within the first rod passageway.

26. The kit of claim 4, which further comprises a threaded set screw, wherein said means for preventing clamping includes a travel stop located in said head, said head includes a threaded portion to receive said set screw, and tightening of said set screw to the travel stop results in clearance of said rod within the first rod passageway.

27. The kit of claim 4, further comprising a set screw threadably received by said second head, wherein tightening of said set screw results in frictional clamping of a rod by a second head.

28. The kit of claim 4, further comprising a set screw threadably received by said second head, wherein tightening of said set screw results in application of a compressive load against the surface of said rod.

29. The kit of claim 4, further comprising a set screw threadably received by said second head, wherein tightening of said set screw can cause frictional clamping of the rod by the second head.

30. The kit of claim 4, wherein a hardening coating of amorphous diamond-like coating is on the rod.

31. The method of claim 5, wherein coating the surface of the prepared portion at the one end with a material adapted and configured to harden the surface includes applying a hardening coating of amorphous diamond-like coating to the rod.

32. The apparatus of claim 2, wherein at least one of the entrance of the rod pathway, the exit of the rod pathway, and the central passage of the rod pathway includes a hard coating.

33. The apparatus of claim 2, wherein at least one of the rounded end of the bone connection device and the pocket of the head receiving therein said bone connection device includes a hard coating.
